(19) United States
(12) Patent Application Publication
(10) Pub. No.: US 2009/0318882 A1
(43) Pub. Date: Dec. 24, 2009

(54) INTRAOSSEOUS DENTAL ANESTHESIA

(76) Inventor: Robert Adler, Lawrence, NY (US)

Correspondence Address: KATTEN MUCHIN ROSENMAN LLP, 575 MADISON AVENUE, NEW YORK, NY, 10022-2585, US

(21) Appl. No.: 12/092,358

(22) PCT Filed: Nov. 21, 2006
(86) PCT No.: PCT/US06/45116

§ 371 Date: Oct. 21, 2008

Related U.S. Application Data
(60) Provisional application No. 60/738,498, filed on Nov. 21, 2005.

(51) Int. Cl. A61K 31/38; A61P 25/04; A61M 19/00

(52) U.S. Cl. 604/272; 514/448

(57) ABSTRACT

A method and kit for intraosseous dental anesthesia that achieves enhanced anesthesia is provided. The method involves delivering 10% or more of a caepule of Articaine HCL with epinephrine to the level of the cortical plate of bone in both the mesial and distal areas of each tooth with a small gauge needle together with pressure and penetrating the cortical plate of bone with the small gauge needle. Where impenetrable, the Articalne HCL with epinephrine is allowed to passively pass through the cortical plate of bone. A method of intraosseous dental anesthesia in cases of acute pulpitis and for tooth extraction together with intraligamental injection technique is also provided.

### CROSS REFERENCE TO RELATED APPLICATIONS

This application claims priority from U.S. Provisional Application No. 60/738,498 filed on Nov. 21, 2005, incorporated herein by reference.

### FIELD OF THE INVENTION

The invention relates generally to dental anesthesia and, more particularly, to a method for intaosseous dental anesthesia.

### BACKGROUND OF THE INVENTION

Usually, an anesthetic is injected into the lingual aspect of the mandibular ramus, using conduction anesthesia, i.e., the mandibular block. Disadvantages of the nerve block technique are increased risk of traumatisation of the nerve trunk and accidental intravascular injection of the local anesthetic solution.

Known means of supplying supplemental injections of an anesthetic are by periodontal ligament injection and intraosseous injection. When mandibular block or infiltration anesthesia is not sufficiently effective, the periodontal ligament injection using a ligamental gun syringe is the next choice. This process suffers from having a short effective period of the anesthesia and the process poses a risk of osteomyelitis.

The other choice is intraosseous injection. Intraosseous injection for dental anesthesia involves injection of the anesthetic composition into the cancellous bone of the alveolar process surrounding the root of the targeted tooth in the inside of the mandible. The concept of intraosseous dental anesthesia has existed for nearly 100 years, albeit with poor results.

Types of intraosseous injections vary. One form of intraosseous injection is the Stabident System by Fairfax, Dental, Inc., which uses a two-step perforation and injection process. This method is described in U.S. Pat. No. 5,173,050 to Dillon in 1992 for methods for drilling an initial perforation, thus allowing the dentist to remove the drill bit and re-enter the passageway with a hypodermic needle to deliver anesthesia. Specifically, the Stabident System consists of using a perforator (0.9 mm long with a 0.43 mm diameter) to drill a hole in the cortical plate of the alveolar process and a separate injection needle (27 gauge) to inject the anesthetic into the cancellous bone within. The injection needle attaches to a separate standard syringe. Kits are available with injection needles modified such that the bevel at the end of the needle has a blunted tip. Generally, the site of a lateral injection for this system is a point about 2 mm apical to the intersection of a horizontal line along the gingival margins of the teeth and a vertical line through the interdental papilla. Unfortunately, this two-step Stabident process presents a risk of damaging the bone while drilling and a risk of drilling into the root surface of the tooth. In addition to the risk of injury to the patient, bleeding and clotting at the drilling site impair the view of the drill point, making precise drilling more difficult and obscuring the opening during the second step of injecting the anesthetic. Furthermore, in a case of periodontal gingival disease, a drill hole cannot be made in the area to be injected. This method was also limited by failures of re-entry.

An improvement over the two-step intraosseous injection process is described in U.S. Pat. No. 3,976,070 to Dumont, which claims a support device for a small gauge hypodermic needle that allows the injection to be completed in a one-step process. The support device includes a funnel-like structure adapted to fit over the base of the hypodermic needle and a sliding tip guard adapted to slide coaxially inside the funnel-like structure in the manner of an expansible telescope. At the end of the tip guard, away from the funnel-like structure, there is an enlarged hub adapted to press against the gum in the mouth of the patient and through which the hypodermic needle is inserted, through the gum and into the bony structure below. This one-step intraosseous needle eliminates the problems associated with the two-step process. The small size of the needle reduces the risk of damage to the bone and the underlying tooth, even if the site for the injection is not accurately located. The proper location for the injection is visually selected at a approximately 1 mm above the alveolar crest of a maxillary tooth or 1 mm below the aveolar crest of a mandibular tooth. Slight miscalculation of the perforation and injection site with this method, makes the anesthetic ineffective on the area where it is needed. Moreover, even if the correct perforation is selected, the needle may bend, buckle, and/or slide along the cortex rather than penetrating the precise selected location, resulting in distorted infusions of the anesthetic and ineffective injection.

U.S. Pat. No. 5,762,639 by Gibbs, and U.S. Pat. No. 5,779,708 by Wu, both described methods to leave the intraosseous hollow channel in place subsequent to drilling as a type of guidance for re-entry. These methods have the disadvantage of breakage of the hollow instruments, the requirement that the drill be left in the jawbone while dental work is conduced, and the possibility of microbial introduction into the underlying tissues.

U.S. Pat. No. 5,432,824 to Akerfeldt, et al discloses a method of accessing a hard tissue whereby a needle drill is inserted into and through a cannula and then used to drill a hole. After the hole is drilled, a cannula is inserted into the oversized hole. The problem with this method is that the cannula is only loosely fitted in the drilled hole, and injected anesthesia may leak backwards out of the hard tissue.

Advantages of intraosseous delivery of dental anesthetic include a very quick onset of anesthesia (approximately 30 seconds) with a significant reduction in the amounts of anesthetic to be used, and profound anesthesia without numbing the tongue and lips. Numbness is limited only to the tooth to be treated. Less time is spent waiting for anesthesia and restorative work can begin almost immediately after injection. Additionally, bilateral mandibular anesthesia can be achieved. Unfortunately, using present intraosseous dental anesthesia techniques, profound anesthesia is not always achieved and patients may experience pain.

Recently, several companies have introduced a technique which drills or bores a hole through the bone in order to increase the efficacy of this injection technique. When the anesthetic is injected in the bone through a hole in the cortical plate, the tissue will not affect it, and it anesthetizes only the area of treatrnent, not the quadrant. Topical anesthetic is applied first and a small amount of anesthetic is injected into the mucous membrane in the area to be perforated. A perforator attached to a high speed drill bores a hole of 0.43 mm through the cortical plate. A needle of 0.4 mm (27 gauge) is inserted into the hole and less than 1 cartridge of anesthetic is delivered into the hole. One cartridge of anesthetic is the maximum per visit using this technique. The needle must be placed in the prepared hole at the same angulation and depth or the anesthesia will leak out with no resulting anesthesia. A hand-on course is recommended for this technique as improper angulation of the perforator can pierce a root.

If the area undergoing surgical manipulation is inflamed or if the patient present with pain, the success rate for profound anesthesia using current intraosseous techniques is diminished further. This is because infected tissue tends to be acidic and acidic tissue does not anesthetize easily.

Therefore, intraosseous anesthesia requires specialized equipment, is very cumbersome as it is technique-sensitive, and sounds barbaric to most patients Despite its advantages, intraosseous injection has not become popular for the reason that there is no practical technique of making the injections successfully. Intraosseous dental anesthesia has remained as merely an alternative when nerve block and infiltration methods fail.

Accordingly, there is a need for an intraosseous dental anesthesia technique which is highly effective and which does not require special equipment and which overcomes some or all or the previously delineated drawbacks of prior intraosseous methods.

### SUMMARY OF THE INVENTION

It is a first object of the present invention to provide a dental intraosseous technique (i.e., the Adler technique) for dental anesthesia which does not require any specialized equipment not routinely found in a dental office, which has an immediate onset and which is effective approximately 95% of the time, for every type of dental procedure.

It is also an object of the present invention to provide a kit directed to same.

It is also an object of the present invention to provide a method of intraosseous dental anesthesia involving the steps of:
1) applying topical anesthetic to the outer epithelial layer;
2) delivering Articaine HCL with epinephrine to the level of the cortical plate; and
3) delivering 10% or more of a carpule of anesthetic in the mesial and distal areas of each tooth.

It is another object of the present invention to provide an improved method for intraosseous injection which may be used instead of, and eliminates the need for, application of the mandibular or mental nerve block. Major nerve blocks such as the mandibular nerve block can cause paresthesia, require the use of longer needles which may cause the operator to “miss” the block, and are somewhat more dependent on local anatomic variations in the patient's anatomy. The bone is more dense around the mandibular apecies, which inhibits the diffusion of the anesthetic. The anatomical variations from patient to patient can make a textbook-perfect injection ineffective.

It is yet another object of the present invention to provide a method for intraosseous injection which is virtually pain free, and targets administration of the local anesthetic to one or several particular teeth. Moreover, the patent experiences almost no numbness of the tongue or lip. There is no ballooning of the soft tissue in the method and post-operative pain is unusual. Four quadrants of dentistry can be performed at one sitting, with no discomfort to the patient.

It is yet another object of the present invention to provide a method for intraosseous intra-pulpal anesthesia.

It is yet another object of the present invention to provide a method for intraosseous anesthesia for extractions which may be supplemented with an intraligamental injection technique.

These and other features, aspects, and advantages of the present invention will become apparent from a reading of the following detailed description of the preferred embodiment of the invention.

### DETAILED DESCRIPTION OF THE PREFERRED EMBODIMENTS

Embodiments of the present invention provide an improved method for intraosseous dental anesthesia.

As used herein the term “intraosseous anesthesia” refers to local administration of an anesthesia into the jawbone itself, as contrasted to soft tissue or into the ligament/septum.

The teeth are imbedded in the jaw in a foundation of cancellous bone. This bone is not dense (or solid), but rather exhibits many open spaces. Above the cancellous bone, outwardly, toward the outside surface of the jaw, are four tissue layers. The outmost layer is the epithelium, followed by connective tissue, the periosteum, and finally the cortical bone. All these serve to cover the cancellous bone. The outer three layers can easily be anesthetized with a small application of a topical anesthetic of about 10-60 seconds. The fourth layer, the cortical bone, is solid bone which is not endowed with any sensory innervation, so it has no feeling when manipulated.

According to the method of intraosseous dental anesthesia of the present invention, there is provided a technique involving the steps of:
1) applying topical anesthetic to the outer epithelial layer;
2) delivering Articaine HCL with epinephrine to the level of the cortical plate; and
3) delivering 10% or more of a carpule of anesthetic at the level of the cortical plate.

This is done with a standard short small gauge needle, such as a 30-gauge needle. Pressure should be applied to the cortical bone with the small gauge needle. Approximately, 85% of the time it will actually perforate the cortical bone. Perforation of the cortical bone is preferred so that the anesthetic solution can be injected into the cancellous bone space around the tooth. However, in 15% of the cases the cortical bone will be impenetrable by the needle. The Articaine HCL with epinephrine based anesthetic will be strong enough to passively pass through the cortical plate of bone into the cancellous bone on its own to achieve profound anesthesia.

A 30 gauge, short needle is preferred to decrease the risk of a hematoma. However, nothing herein is meant to limit use to 30 gauge, short needles. Other short needles, including but not limited to, 25 or 27 gauge short or ultrashort may also be used. The injection needle is preferably constructed of high strength material to resist bending and buckling when penetrating cortical bone.

The present inventor has surprisingly found that successful anesthesia depends on the technique of injection. Correct positioning of the needle for perforation and injection is crucial. The site of insertion of the needle is both mesial and distal to each tooth, approximately 3 mm apical to the gingival crest. After proper tissue preparation with antiseptic and topical, the bevel should be toward the bone and the needle inserted. The depth will vary according to the anatomy of the patient.

By the term carpule as used herein is meant cartridges. Carpule is a trade name for cartridges (Cook-Waite Laboratories). The carpules (cartridges) that dental local anesthetics are distributed in contain 1.8 ml of solution. Articaine (Septocaine®) carpules contain 1.7 ml. Carpules contain local anesthetic, sodium chloride, distilled water and a vasoconstrictor drug with preservative.

A carpule of 4% Articaine (Septocaine®) by Septodont) contains 40 mg of anesthetic agent per milliliter which means that each 1.7 ml cartridge contains 72 mg of agent.

It is understood that other local anesthetics composed of an aromatic nucleus connected to an ester or an amide linkage to an aliphatic chain containing a secondary or a tertiary amino group capable of penetrating or diffusing into bone may be used. The local anesthetic agent must have a high intrinsic activity and a low systemic toxicity. Examples of suitable local anesthetic agents are lidocaine, xylocaine, mepivacaine, prilocaine, chloroprocaine, propoxycaine, dibucaine, bupivacaine, etidocaine, ropivacaine, and articaine or combinations thereof. A preferred anesthetic is Articaine.

Anesthesia from the current invention has an immediate onset and a duration of action of up to two hours. Articaine differs from other local anesthetic agents in that it has a higher local anesthetic efficacy and a lower systemic toxicity. The relation of local anesthetic efficacy and systemic toxicity is the best of all local anesthetics, due to high plasma protein binding rate and fast metabolism.

Commercially available Articaine is available from Septodont. Articaine HCL with epinephrine (e.g. Septocaine® by) (Zorcaine® by Cook-Waite). Other commercially available local anesthetics include Carbocaine® (mepivicaine) and Citanest® (prilocaine).

The anesthetic composition further comprises vasoconstrictors to decrease blood flow at the site of injection to both prolong the duration of the anesthetic agent and to decrease the rate of absorption from the site of administration in order to reduce the potential systemic toxicity. The result of such decreased blood flow is a decrease in toxicity and prolongation of the duration of anesthesia. The vasoconstrictor should have a high alpha-adrenergic activity to ensure an effective vasoconstrictive activity even in a low concentration. Examples of suitable vasoconstrictors are epinephrine, norepinephrine, levonordefrin, phenylephrine, and felypressin. A preferred vasoconstrictor is epinephrine.

The concentration of vasoconstrictor depends on the duration of anesthesia desired. The higher the concentration, the longer the duration. Preferably, the concentration of the vasoconstrictor in the anesthetic composition is from about 0.005 mg/ml (i.e. 5 mcg/ml or 1:200,000) to about 0.04 mg/ml (40 mcg/ml or 1:25,000). Preferred is a concentration of 1:100,000 (0.01 mg/ml or 10 mcg/ml).

Optionally, the anesthetic composition contains an antibacterial agent; an antifungal agent; a preservative such as methylparaben; preservatives for the vasoconstrictor agents such as sodium bisulfite or metabisulfite; diluents, or combinations thereof.

The quantity of anesthetic to be injected is about 10% to about 100% of the carpule in the mesial and about 10% to about 100% of the carpule in the distal area. This may be between from about 0.17 ml to about 5 ml depending on the concentration of the anesthetic agent and other factors such as age of the patent. If profound anesthesia is not achieved, the procedure may be repeated.

In another embodiment, in cases of acute pulpitis, the method may additionally involves introduction of anesthetic directly into the canal using an intra-pulpal technique. Although intraosseous injection would usually provide sufficiently profound anesthesia to enable the operator to uncover the pulpal floor of the acutely painful tooth, once the pulpal floor is uncovered and the canals are exposed, it may be necessary to inject directly into the canal. This is achieved by insertion of a small gauge needle directly into the canal, and forcefully injecting the solution. With the intra-pulpal technique, the patient might experience one second of sensation, followed by profound anesthesia.

By intra-pulpal technique as used herein is meant injection of a local anesthetic into the cancellous bone of the alveolar process surrounding a targeted tooth that requires anesthesia prior to endodontic treatment.

In one embodiment, the intraesseous technique is used for extractions. Here the method is supplemented with an intraligamental injection technique. According to this technique there is deposited about 10-100% of a carpule of anesthetic, with a small gauge needle, such as a 30 gauge needle slightly bent, for example by 45°, into the four corners of the tooth, into the space between the actual tooth structure and the soft tissue including the mesio-buccal corner, the mesio-distal corner, the mesio-lingual corner, and the distolingual corner.

This method allows the operator to work on teeth in all four quadrants in one sitting, under a completely painless condition, with almost no numbing sensation of the lips or tongue.

The method of the present invention is simple to administer. The operator does not have to follow small anatomical landmarks, as would be necessary in a block. Additionally, there is no danger of inadvertently hitting and damaging a blood vessel or nerve, as in a block.

Four quadrants of dentistry can be performed at one sitting, with no discomfort to the patient. This is especially significant in those cases where total anesthetic dose is a concern, e.g. significant liver disease. In such patients only one quadrant can be worked on at a time.

In another embodiment, there is provided a dental kit for intraosseous dental anesthesia comprising: a) a carpule of Articaine 4% with epinephrine 1:100,000; b) a short 30 gauge needle; and c) instructions for the “Adler technique”.

The following example describes specific aspects of the invention to illustrate the invention and provide a description of the present methods for those skilled in the art. The Examples should not be construed as limiting the invention as the examples merely provide specific methodology useful in the understanding and practice of the invention and its various aspects.

### EXAMPLE 1

Each tooth is tested before injection of local anesthetic to establish its baseline response, and each two minutes after injection until the absence of sensation to the maximal stimulas (latency time). After this period it is tested every 10 minutes until the return to the base threshold level.

A volume of 0.5 ml of Articaine 2% with epinephrine 1:100,000 is injected into the mesial and distal areas of tooth number 32 of healthy subjects. The efficacy of the anesthetic is then verified by application of electric stimulus, Vitality Scanner Analytic Technology pulp tester. It is concluded that Articaine injected via the Adler technique results in anesthetic efficacy in 97% of the subjects.

While certain preferred and alternative embodiments of the invention have been set forth for purposes of disclosing the invention, modification to the disclosed embodiments can occur to those who are skilled in the art.

We claim:

1. A method for intraosseous dental anesthesia comprising the steps of:
1) applying topical anesthetic to the outer epithelial layer;
2) delivering 10% or more of a carpule of Articaine HCL with epinephrine to the level of the cortical plate of bone in both the mesial and distal areas of each tooth with a small gauge needle;
3) applying pressure to the cortical plate of bone with the small gauge needle; and
4) either penetrating the cortical plate of bone with the small gauge needle or allowing the Articaine HCL to passively pass through the cortical plate of bone.

2. The method for intraosseous dental anesthesia of claim 1, wherein the Articaine is present in a concentration ranging from 2-4%.

3. The method for intraosseous dental anesthesia of claim 2, wherein the Articaine is present in a concentration of 4%.

4. The method for intraosseous dental anesthesia of claim 1, wherein the epinephrine is present in a concentration ranging from 1:200,000 to 1:25,000.

5. The method for intraosseous dental anesthesia of claim 4, wherein the concentration of epinephrine is present in a concentration of 1:100,000.

6. The method for intraosseous dental anesthesia of claim 1, wherein the concentration of Artaine with epinephrine is 4% and 1:100,000, respectively.

7. The method for intraosseous dental anesthesia of claim 1, wherein the small gauge needle has a gauge of between 25 to 30.

8. The method for intraosseous dental anesthesia of claim 7, wherein the small gauge needle has a gauge of 30.

9. The method for intraosseous dental anesthesia of claim 1, wherein the small gauge needle is short or ultra-short.

10. A method for intraosseous intra-pulpal anesthesia comprising the steps of:
1) applying topical anesthetic to the outer epithelial layer;
2) delivering 10% or more of a carpule of Articaine HCL with epinephrine to the level of the cortical plate of bone in both the mesial and distal areas of each tooth with a small gauge needle;
3) applying pressure to the cortical plate of bone with the small gauge needle;
4) either penetrating the cortical plate of bone with the small gauge needle or allowing the Articaine HCL to passively pass through the cortical plate of bone; and
5) injecting the Articaine with epinephrine directly into the canal.

11. A method for intraosseous anesthesia for dental extraction used together with intraligamental injection technique comprising the steps of:
1) applying topical anesthetic to the outer epithelial layer; and
2) delivering 10% or more of a carpule of Articaine HCL with epinephrine with a slightly bent small gauge needle into the four comers of the tooth, into the space between the tooth structure and the soft tissue.

12. The method of claim 1, wherein said small gauge needle is bent to approximately 45°.

13. The method of claim 1, further comprising a second local anesthetic selected from the group consisting of lidocaine, mepivacaine, prilocaine, chloroprocaine, procaine, propoxycaine, dibucaine, bupivacaine, ropivacaine, and etidocaine, or a combination thereof.

14. A dental kit for intraosseous dental anesthesia comprising:
a) a carpule of Articaine 4% with epinephrine 1:100,000;
b) a short 30 gauge needle; and
c) instructions for the “Adler technique”.
